(19) United States
(12) Patent Application Publication
(10) Pub. No.: US 2008/0241829 A1
(43) Pub. Date: Oct. 2, 2008

(54) Methods And Kits For Producing Labeled Target Nucleic Acid For Use In Array Based Hybridization Applications

(76) Inventors: Stephen B. Milligan, Los Altos, CA (US); Eric M. Leproust, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/695,201

(22) Filed: Apr. 2, 2007

(51) Int. Cl. C12Q 1/68

(52) U.S. Cl. 435/6

(57) ABSTRACT

Methods for producing labeled probe nucleic acids from genomic nucleic acid template are provided. In some embodiments of the subject methods, a plurality of sequence-specific primers are employed to enzymatically generate a set of labeled target nucleic acids corresponding to coding regions of genes from a genomic template via a primer extension protocol. The subject methods find use in a variety of different applications, and can be used, for example, in the preparation of labeled probe nucleic acids for use in array based comparative genomic hybridization applications. Also provided are kits for use in practicing the subject methods.

### BACKGROUND

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. Identification of the genetic events leading to neoplastic transformation and subsequent progression can facilitate efforts to define the biological basis for disease, improve prognostication of therapeutic response, and permit earlier tumor detection. In addition, perinatal genetic problems frequently result from loss or gain of chromosome segments such as trisomy 21 or the micro deletion syndromes. Thus, methods of prenatal detection of such abnormalities can be helpful in early diagnosis of disease.

Comparative genomic hybridization (CGH) is one approach that has been employed to detect the presence and identify the location of amplified or deleted sequences. CGH reveals increases and decreases irrespective of genome rearrangement. In one implementation of CGH, genomic DNA is isolated from normal reference cells, as well as from test cells (e.g., tumor cells). The two nucleic acids are differentially labeled and then hybridized in situ to metaphase chromosomes of a reference cell. The repetitive sequences in both the reference and test DNAs are either removed or their hybridization capacity is reduced by some means. Chromosomal regions in the test cells which are at increased or decreased copy number can be identified by detecting regions where the ratio of signal from the two DNAs is altered. For example, those regions that have been decreased in copy number in the test cells will show relatively lower signal from the test DNA than the reference compared to other regions of the genome. Regions that have been increased in copy number in the test cells will show relatively higher signal from the test DNA.

In a recent variation of the above traditional CGH approach, the immobilized chromosome element has been replaced with a collection of solid support bound target nucleic acids, e.g., an array of cDNAs. Such approaches offer benefits over immobilized chromosome approaches, but introduce new problems. For example, only a small percentage of the genome is represented in the collection of solid support bound targets and, therefore, only a small percentage of the labeled probe material actually hybridizes to the immobilized targets, which can result in low signal intensities for genomic derived probe nucleic acids populations.

Accordingly, there is interest in the development of improved array based CGH protocols.

### SUMMARY

Methods and kits for producing labeled target nucleic acids from genomic nucleic acid template are provided. In some embodiments, a sequence-specific primer is employed to enzymatically generate a labeled target nucleic acid via primer extension. In some embodiments, a probe-binding sequence within the target that is complementary to a sequence within a selected region, such as a coding region, of one strand of the genomic template. The probe-binding sequence is complementary to a probe on a microarray.

In some embodiments, the sequence-specific primer recognizes a selected sequence within, or adjacent to a coding region in the genomic template. The primer is designed based on the known sequence of the genomic template. The primer is designed to bind to the template such that the 3′ end of the primer is positioned downstream from the selected region in the template.

A plurality of primers can be designed for use in a plurality of primer extension reactions of a selected region of a template. In some embodiments, a plurality of primers (e.g., between 1 to 100 primers, or between 1 to 20 primers) can be used in the primer extension procedure. The primers can be designed to avoid cross-binding, i.e., overlapping binding between primers. The primers can be designed such that the probe-binding sequences have similar melting temperatures. A plurality of primers can be designed for use in simultaneous primer extensions of a plurality of different selected regions of a genomic template.

Also provided are kits for use in practicing the subject methods. Kits can comprise one or more of the following: a plurality of sequence-specific primers as described herein, a microarray comprising a plurality of probes for binding to probe-binding regions in targets generated during primer extension of said primers, and a nucleic acid polymerase suitable for use in the above methods.

The subject methods result in a lower complexity labeled target population which results in higher signal and improved signal to background. The subject methods and kits find use in a variety of different applications, and can be used, for example, in the preparation of labeled probe nucleic acids for use in array based comparative genomic hybridization applications.

### BRIEF DESCRIPTION OF THE FIGURES

FIG. 1 provides a diagram of some embodiments of a protocol for preparing labeled nucleic acid targets.

FIG. 2 provides a schematic representation of methods employing random primers.

FIG. 3 provides a schematic representation of the use of sequence-specific primers.

FIG. 4 provides a schematic representation of some embodiments of the use of sequence-specific primers.

### DESCRIPTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that, as used in the specification and the appended claims, the singular forms “a,” “an” and “the” include plural referents unless the context clearly dictates otherwise. Thus, for example, reference to “a solid support” includes a plurality of solid supports. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range, and any other stated or intervening value in that stated range, is encompassed within the description. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included in the present disclosure.

Unless defined otherwise, all technical and scientific terms used herein have the same meaning as commonly understood to one of ordinary skill in the art to which this invention belongs. Various methods, devices and materials similar or equivalent to those described herein can be used in the practice or testing of the disclosed methods.

All publications mentioned herein are incorporated herein by reference for the purpose of describing and disclosing the invention components that are described in the publications which might be used in connection with the present disclosure.

As summarized above the present disclosure provides methods of producing labeled nucleic acids from genomic template nucleic acid using a primer or set of primers, as well as kits for use in practicing the subject methods. The subject methods are discussed first in greater detail, followed by a review of representative kits for use in practicing the subject methods.

### DEFINITIONS

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible that methods recited herein may be carried out in any order of the recited events which is logically possible, as well as the recited order of events.

In this specification and in the claims that follow, reference will be made to a number of terms that shall be defined to have the following meanings unless a contrary intention is apparent.

The term “nucleic acid” as used herein means a polymer composed of nucleotides, e.g. deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The term “functionalization” as used herein relates to modification of a solid substrate to provide a plurality of functional groups on the substrate surface. By a “functionalized surface” as used herein is meant a substrate surface that has been modified so that a plurality of functional groups are present thereon.

The terms “reactive site”, “reactive functional group” or “reactive group” refer to moieties on a monomer, polymer or substrate surface that may be used as the starting point in a synthetic organic process. This is contrasted to “inert” hydrophilic groups that could also be present on a substrate surface, e.g., hydrophilic sites associated with polyethylene glycol, a polyamide or the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using functionalized substrates as described herein, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In some embodiments, oligomers will comprise about 2-50 monomers, about 2-20, or about 3-10 monomers. In some embodiments, oligomers will comprise about 2-500 monomers, about 2-200, or about 3-100 monomers.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “oligonucleotide bound to a surface of a solid support” refers to an oligonucleotide or mimetic thereof, e.g., PNA, that is immobilized on a surface of a solid substrate in a feature or spot, where the substrate can have a variety of configurations, e.g., a sheet, bead, or other structure. In some embodiments, the collections of features of oligonucleotides employed herein are present on a surface of the same planar support, e.g., in the form of an array.

The term “array” encompasses the term “microarray” and refers to an ordered array presented for binding to nucleic acids and the like. Arrays, as described in greater detail below, are generally made up of a plurality of distinct or different features. The term “feature” is used interchangeably herein with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids.

An “array” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm or even less than 10 cm², e.g., less than about 5 cm², including less than about 1 cm², less than about 1 mm², e.g., 100μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In some embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. The substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

In some embodiments, in situ prepared arrays are employed. In situ prepared oligonucleotide arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water, as known in the art and further described in copending application Ser. No. 10/449,838, the disclosure of which is herein incorporated by reference. Other features of in situ prepared arrays that make such array formats of particular interest in some embodiments of the present invention include, but are not limited to: feature density, oligonucleotide density within each feature, feature uniformity, low intra-feature background, low inter-feature background, e.g., due to hydrophobic interfeature regions, fidelity of oligonucleotide elements making up the individual features, array/feature reproducibility, and the like. The above benefits of in situ produced arrays assist in maintaining adequate sensitivity while operating under stringency conditions required to accommodate highly complex samples.

In selecting probes, it can be useful to use a computational algorithm to produce a calculated melting temperature for each probe. Sets of probe that have a narrow melting temperature range may be particularly suited for some applications of array hybridization analysis. A nearest neighbor analysis that adjusts for mismatches in the probe sequences can be used to generate the calculated melting temperatures. In an embodiment with no mismatches, a simpler nearest neighbor algorithm can be used. Software methods for calculating melting temperatures are well developed, and such may be obtained from various commercial or academic sources. Some commercial sources for software include Alkami Biosystems, Molecular Biology Insights, PREMIER Biosoft International, IntelliGenetics Inc., Hitachi Inc., DNA Star, Advanced American Biotechnology and Imaging. Various references have described melting temperature calculations, including Breslauer et al., “Predicting DNA duplex stability from the base sequence”, Proc Natl Acad. Sci. (1986) 83:3746-3750; Sugimoto et al., “Improved thermodynamic parameters and helix initiation factor to predict stability of DNA duplexes” Nucleic Acids Research (1996) 24:4501; Xia et al., “Thermodynamic parameters for an expanded nearest-neighbor model for formation of RNA duplexes with Watson-Crick base pairs” Biochemistry (1998) 37(42): 14719-35; and references therein.

Probes may be selected, e.g. based on sequence, GC content, AT content, location in the genome, or based on empirical performance in use, or based on other appropriate factors. In some embodiments, the calculated melting temperatures of at least about 80% of the probes on an array fall within a range of about 6 degrees Celsius. In some embodiments, the calculated melting temperature of each probe is obtained using a nearest neighbor analysis algorithm and the genomic template sequence that the probe is directed to, and may include any insertions, deletions, or substitutions. It is further noted that the particular methodology used to select probe sets is illustrative only, and should not be interpreted to limit the scope of the disclosure.

In some embodiments, the oligonucleotides that make up the distinct features are ones that have been designed according to one or more particular parameters to be suitable for use in a given application, where representative parameters include, but are not limited to: length, melting temperature (Tm), non-homology with other regions of the genome, signal intensities, kinetic properties under hybridization conditions, etc., see e.g., U.S. Pat. No. 6,251,588, the disclosure of which is herein incorporated by reference. In some embodiments, the entire length of the feature oligonucleotides is employed in hybridizing to sequences in the genome, while in some embodiments, only a portion of the immobilized oligonucleotide has sequence that hybridizes to sequence found in the genome of interest, e.g., where a portion of the oligonucleotide serves as a tether. For example, a given oligonucleotide may include a 30 nt long genome specific sequence linked to a 30 nt tether, such that the oligonucleotide is a 60-mer of which only a portion, e.g., 30 nt long, is genome specific.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably. By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO₄, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In some embodiments, the stringency of the wash conditions set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

Some embodiments of stringent assay conditions comprise rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions include hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Stringent hybridization conditions can also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

As used herein, the term “primer” refers to a polynucleotide that is capable of hybridizing (i.e., annealing) with a polynucleotide and serving as an initiation site for nucleotide polymerization. “Primer extension” is the enzymatic addition, i.e., polymerization, of monomeric nucleotide units to a primer while the primer is hybridized (annealed) to a template polynucleotide. Primer extension is initiated at the template site where a primer anneals.

The phrase “labeled population of nucleic acids”, or “labeled polynucleotides”, or other such language refers to mixture of nucleic acids that are detectably labeled, e.g., fluorescently labeled, such that the presence of the nucleic acids can be detected by assessing the presence of the label. The labeled population of nucleic acids is “made from” a chromosome source, the chromosome source is usually employed as template for making the population of nucleic acids. In some embodiments, the sample that is hybridized on an array includes reference target and analyte target, wherein the reference target and the analyte target are differentially labeled.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not especially distinct. In other words, a mixture is not addressable. To be specific, an array of surface bound polynucleotides, as is commonly known in the art and described below, is not a mixture of capture agents because the species of surface bound polynucleotides are spatially distinct and the array is addressable.

“Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well known in the art and include, for example, ion-exchange chromatography, affinity chromatography, flow sorting, and sedimentation according to density.

The term “sample” as used herein relates to a material or mixture of materials, typically, although not necessarily, in fluid form, containing one or more components of interest.

“Template” references a polynucleotide, typically from a genome of an organism.

“Complementary” references a property of specific binding between polynucleotides based on the sequences of the polynucleotides. As used herein, polynucleotides are complementary if they bind to each other in a hybridization assay under stringent conditions, e.g. if they produce a given or detectable level of signal in a hybridization assay. Portions of polynucleotides are complementary to each other if they follow conventional base-pairing rules, e.g. A pairs with T (or U) and G pairs with C. “Complementary” includes embodiments in which there is an absolute sequence complementarity, and also embodiments in which there is a substantial sequence complementarity. “Absolute sequence complementarity” means that there is 100% sequence complementarity between a first polynucleotide and a second polynucleotide, i.e. there are no insertions, deletions, or substitutions in either of the first and second polynucleotides with respect to the other polynucleotide (over the complementary region). Put another way, every base of the complementary region may be paired with its complementary base, i.e. flowing normal base-pairing rules. “Substantial sequence complementarity” permits one or more relatively small (less than 10 bases, e.g. less than 5 bases, typically less than 3 bases, more typically a single base) insertions, deletions, or substitutions in the first and/or second polynucleotide (over the complementary region) relative to the other polynucleotide. The region that is complementary between a first polynucleotide and a second polynucleotide (e.g. a target and a probe) is typically at least about 10 bases long, more typically at least about 15 bases long, still more typically at least about 20 bases long, or at least about 25 bases long. The region that is complementary between a first polynucleotide and a second polynucleotide (e.g. target and a probe) may be up to about 200 bases long, or more typically up to about 120 bases long, more typically up to about 100 bases long, still more typically up to about 80 bases long, yet more typically up to about 60 bases long, more typically up to about 45 bases long.

“Upstream” as used herein refers to the 5′ direction along the template. “Downstream” refers to the 3′ direction along the template. Hence, a primer binding downstream of a coding sequence is located at (or is complementary to) a sequence of the template that is in the 3′ direction from the coding sequence site along the template.

Following hybridization and washing, as described above, the hybridization of the labeled nucleic acids to the probes can be detected using standard techniques so that the surface of immobilized probes, e.g., the array, is interrogated, or read. Reading the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent applications: Ser. No. 09/846,125 “Reading Multi-Featured Arrays” by Dorsel et al.; and U.S. Pat. No. 6,406,849. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In some embodiments, results from interrogating the array are used to assess the level of binding of the population of labeled nucleic acids to probes on the array. The term “level of binding” means any assessment of binding (e.g. a quantitative or qualitative, relative or absolute assessment) usually done, as is known in the art, by detecting signal (i.e., pixel brightness) from a label associated with the sample nucleic acids, e.g. the digested sample is labeled. The level of binding of labeled nucleic acid to probe is typically obtained by measuring the surface density of the bound label (or of a signal resulting from the label).

In some embodiments, a surface-bound polynucleotide may be assessed by evaluating its binding to two populations of nucleic acids that are distinguishably labeled. In these embodiments, for a single surface-bound polynucleotide of interest, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

The term “assessing” and “evaluating” are used interchangeably to refer to any form of measurement, and includes determining if an element is present or not. The terms “determining,” “measuring,” and “assessing,” and “assaying” are used interchangeably and include either or both of quantitative and qualitative determinations. Assessing may be relative or absolute. “Assessing the presence of” includes determining the amount of something present, as well as determining whether it is present or absent.

“Sensitivity” is a term used to refer to the ability of a given assay to detect a given analyte in a sample, e.g., a nucleic acid species of interest. For example, an assay has high sensitivity if it can detect a small concentration of analyte molecules in sample. Conversely, a given assay has low sensitivity if it only detects a large concentration of analyte molecules (i.e., specific solution phase nucleic acids of interest) in sample. A given assay's sensitivity is dependent on a number of parameters, including specificity of the reagents employed (e.g., types of labels, types of binding molecules, etc.), assay conditions employed, detection protocols employed, and the like. In the context of array hybridization assays, such as those of the present invention, sensitivity of a given assay may be dependent upon one or more of: the nature of the surface immobilized nucleic acids, the nature of the hybridization and wash conditions, the nature of the labeling system, the nature of the detection system, etc.

The acronym “CGH” refers to Comparative Genomic Hybridization.

The acronym “aCGH” refers to microarray-based CGH.

Although much of the description herein is directed at aCGH applications, the invention is not limited to methods of array hybridization for aCGH applications, and can be used in other applications such as, for example, methylation analysis, and expression analysis.

Methods

In some embodiments, there are provided methods for generating labeled target nucleic acids from a genomic template, where a feature of the subject methods is the use of a sequence-specific primer in a primer extension protocol.

In practicing the subject methods, the first step is to provide a genomic template. By genomic template is meant the nucleic acids that are used as template in the primer extension reactions as described herein. In some embodiments, the genomic template is a population of genomic deoxyribonucleic acid molecules, whereby population is meant a collection of molecules in which at least two constituent members have nucleotide sequences that differ from each other, e.g., by at least about 1 basepair, by at least about 5 basepairs, by at least about 10 basepairs, by at least about 50 base pairs, by at least about 100 base pairs, by at least about 1 kb, by at least about 10 kb etc.

In some embodiments, the number of distinct sequences in a population of molecules making up a given genomic template can be at least 2, at least 10, or at least 50, where the number of distinct molecules may be 1000, 5000, 10000, 100000 or higher.

The genomic template can be prepared using any convenient protocol. In many embodiments, the genomic template is prepared by first obtaining a source of genomic DNA, e.g., a nuclear fraction of a cell lysate, where any convenient means for obtaining such a fraction may be employed and numerous protocols for doing so are well known in the art. The genomic template may be genomic DNA representing the entire genome from a particular organism, tissue or cell type or may comprise a portion of the genome, such as a single chromosome. Genomic template may be prepared from a subject, for example a plant or an animal, that is suspected of being homozygous or heterozygous for a deletion or amplification of a genomic region. In many embodiments, the average size of the constituent molecules that make up the genomic template do not exceed about 10 kb in length, typically do not exceed about 8 kb in length and sometimes do not exceed about 5 kb in length, such that the average length of molecules in a given genomic template composition may range from about 1 kb to about 10 kb, usually from about 5 kb to about 8 kb in some embodiments. The genomic template may be prepared from an initial chromosomal source by fragmenting the source into the genomic template having molecules of the desired size range, where fragmentation may be achieved using any convenient protocol, including but not limited to: mechanical protocols, e.g., sonication, shearing, etc., chemical protocols, e.g., enzyme digestion, etc.

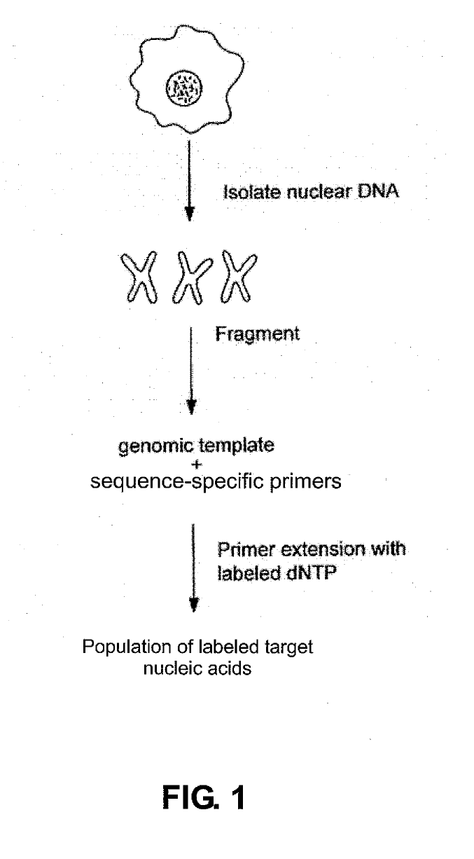

Following preparation, the genomic template is employed in the preparation of labeled target nucleic acids in a protocol in which at least one primer, and often a mixture of different primers, are employed to generate target nucleic acids. A representative protocol is shown in FIG. 1.

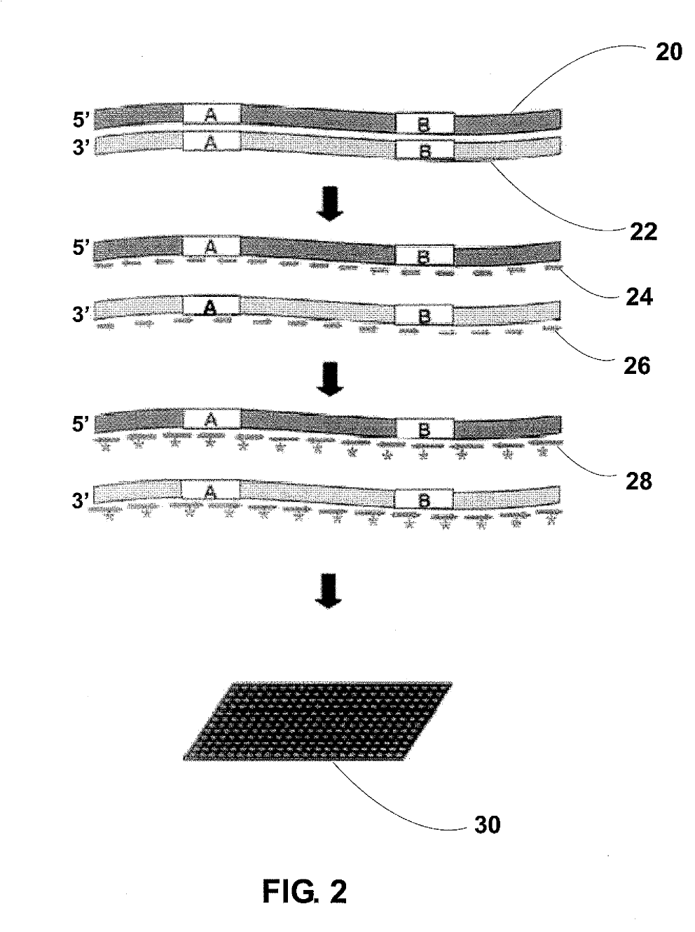

Labeling methods utilizing “random” primers have been described. (See, e.g., U.S. Pat. No. 7,011,949; and U.S. Pat. Publication No. 20040191813. See also, e.g., Bioprime® labeling kit (available from Invitrogen) and derisilab.ucsf.edu/pdfs/GenomicDNALabel.A.pdf.) FIG. 2 exemplifies such procedures in which a double-stranded genomic template (comprising strand 20 and strand 22) is denatured; random primers at 24 and at 26 are annealed to each of the two strands; and labeled target 28 are generated. “A” and “B” represent genes in the genomic DNA. Probes on microarray 30 are designed to hybridize with these targets. Such methods are non-selective in the template DNA that is bound by the primers. Labeled targets are generated that are not represented by compliment probes on the microarray, thus adding to noise in the detected signal due to cross-hybridization. Only a small percentage of the labeled target material actually hybridizes to the immobilize probes, which results in low signal intensities for genomic derived target nucleic acid populations. In addition, the use of random primer in the labeling protocol can generate complimentary target strands which can subsequently hybridize together rendering them insufficient for binding to their cognate array probe. Thus, the high complexity of the labeled target can result in increased noise and decreased probe signal on the microarry.

In contrast, the present methods do not use random primers. From the knowledge of the sequence of the genomic template, a primer can be designed to generate a labeled target corresponding to essentially any desired sequence in the template. In the present methods, the primers are designed such that the target nucleic acids include a sequence of nt residues that are complementary to a sequence within a coding region.

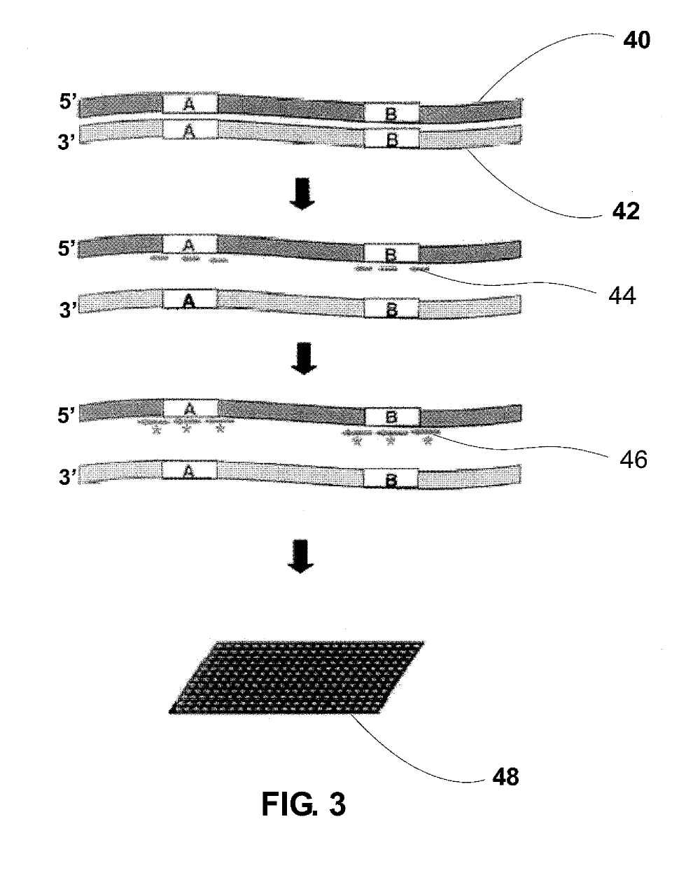

Some embodiments of the present methods are illustrated in FIG. 3 in which strand 40 and strand 42 of a double-stranded genomic template are denatured. Primers, such as primer 44, are annealed and enzymatically extended to form labeled target nucleic acids, such as nucleic acid 46. The primers are designed, as described herein, such that they anneal selectively to the single strand 40, near or within the genomic regions “A” and “B”.

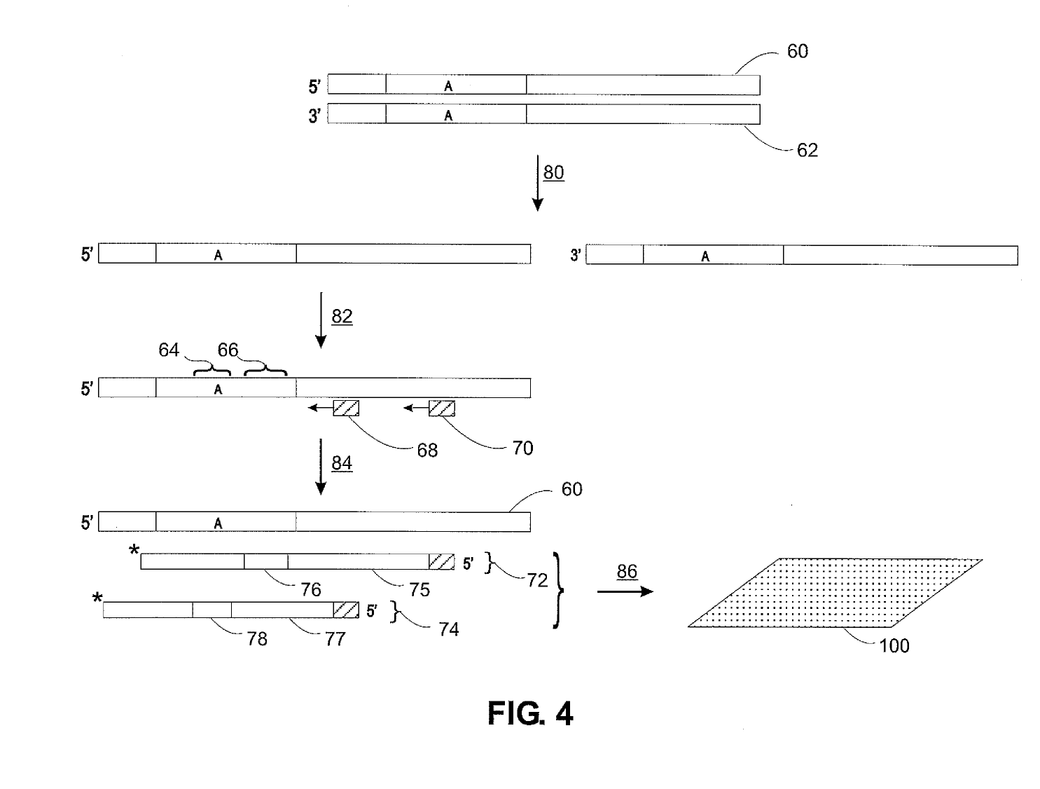

FIG. 4 illustrates some embodiments of the present methods. Double-stranded genomic template, comprising strand 60 and strand 62, is denatured (step 80). In step 82, sequence-specific primer 68 and sequence-specific primer 70 are annealed downstream of coding region “A” which comprises region 64 and region 66. A sequence-specific primer can be designed to bind at any suitable position downstream of the coding region. For example, the primer can bind 1, 10, 20, 50, 100, 500, 1000 or more nt downstream of the coding region. During primer extension 84 in the presence of labeled nucleotide, primer 70 generates labeled target 72, and primer 68 generates labeled target 74. Probe-binding region 76 is complementary to region 66 and also to a probe (not shown) on microarray 100. Probe-binding region 78 is complementary to region 64 and also to a probe on microarray 100. At step 86, a mixture of target 72 and target 74 are separated from strand 60, and hybridized to array 100. The length of intervening sequence 75 and of intervening sequence 77 can be any suitable number of nucleotides. In some embodiments, each intervening sequence can range in length from about 1 to 1000 nt, from about 5 to 500 nt, or from about 10 to 100 nt.

In some embodiments, a microarray for use in the present methods is designed such that there is a complementary probe for each different probe-binding sequence in the target nucleic acids generated in the labeling step. During the hybridization step, each probe-binding sequence hybridizes to its complementary, or substantially complementary, probe on the microarray. For each target, the probe-binding sequence will have the same sequence as the corresponding sequence in the original genomic DNA strand.

The dataset used for designing primers and microarray probes as described herein can be drawn from one or more databases. Exemplary databases containing known biological sequences include the NCBI nt database (ncbi.nih.gov), the TIGR (The Institute for Genomic Research) gene indices (tigr.org/tdb/tgi/index.shtml), the NCBI's Unigene datasets (e.g., for H. sapiens, A. thaliana, and C. elegans) (ncbi.nlm.nih.gov/entrez/query.fcgi?db=unigene), Genebank, and the USCS Genome browser website (genome.ucsc.edu). Those of skill in the art will appreciate that there are also other databases that are available and that contain additional sequences from many different organisms. Publicly available sequence databases include those maintained by: GenBank (Bethesda, Md. USA) (ncbi.nih.gov/genbank/), European Molecular Biology Laboratory's European Bioinformatics Institute (EMBL-Bank in Hinxton, UK) (ebi.ac.uk/embl/), the DNA Data Bank of Japan (Mishima, Japan) (ddbj.nig.acjp/), the Ensembl project (ensembl.org/index.html). Examples of databases that can be obtained and/or searched through the NCBI web portal (ncbi.nih.gov) include Entrez Nucleotides (including data from GenBank, RefSeq, and PDB), all divisions of GenBank, RefSeq (nucleotides), dbEST, dbGSS, dbMHC, dbSNP, dbSTS, TPA, UniSTS, PopSet, UniVec, WGS, Entrez Protein (including data from SwissProt, PIR, PRF, PDB, and translations from annotated coding regions in GenBank and RefSeq), RefSeq (proteins), and many others.

It will be appreciated that some datasets are directed to certain types of sequence information. By way of example, some datasets are directed to genomic sequences, while other datasets are directed to expressed sequences. Still other datasets are directed to polypeptide sequences. The appropriate dataset for use will depend on both the type of array intended (CGH, expression, etc.) and the identity of the organism of interest.

In some embodiments, the sequence-specific primers employed in the subject methods are at least about 6 nt in length. In some embodiments, an oligonucleotide primer employed in the subject methods is one that ranges in length from about 3 to about 25 nt, from about 5 to about 20 nt, from about 10 to about 50 nt, from about 5 to about 10 nt, or from about 20 to about 100 nt. By sequence-specific primer is meant an oligonucleotide that is complementary to a known sequence in the genomic sequence. In some embodiments, the sequence-specific primers are designed to anneal such that they extend into a selected coding region of the gene (exon) during primer extension. A sequence-specific primer can be designed to anneal to a sequence adjacent to a coding region (e.g., downstream of a coding region), within a coding region, or to overlap the junction of a coding region and non-coding region. Primers used in the present methods are devoid of indeterminate nucleotides or random sequences.

A plurality of primers can be designed for use in a plurality of primer extension reactions which extend into a region of interest, such as a coding region. In some embodiments, a plurality of primers (e.g., between 10 to 10000 primers, between 1 to 1000 primers, between 1 to 100 primers, or between 1 to 20) can be used in the primer extension procedure. When a plurality of sequence-specific primers are used, the length and composition of each sequence-specific primer can be designed in order to minimize or substantially eliminate interference with the binding of other sequence-specific primers. For example, any cross-binding between primers or overlap of the sequences along the template can be avoided. The primers can be designed such that the probe-binding sequences have similar melting temperatures (e.g., within a defined range, such as 6° C.). A primer (or primers) can be designed for optimal binding during the primer extension reaction; the probe-binding region generated during primer extension, and its complementary microarray probe, can be selected in order to optimize the hybridization to the microarray. A plurality of primers can be designed for use in simultaneous primer extensions of a plurality of different regions (such as coding regions) of a genomic template. In some embodiments, the number of primers can range from 10 primers to 3 million primers or more.

Conventional techniques for primer design can be used. Exemplary references include: Rozen S, Skaletsky H (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386; dnasoftware.com/Science/Publications/index.htm; cbi.pku.edu.cn/mirror/GenomeWeb/nuc-primer.html; SantaLucia, J., Jr. (2006) “Physical Principles and Visual-OMP Software for Optimal PCR Design”, Methods in Molecular Biology: PCR Primer Design, 2006, Anton Yuryev, Ed., Humana Press, Totowa, N.J. (2006) in press; Norman E. Watkins, Jr. and John SantaLucia, Jr. (2005) “Nearest-neighbor thermodynamics of deoxyinosine pairs in DNA duplexes”, Nucleic Acids Research, 2005, Vol. 33, No. 19, 6258-6267; John SantaLucia, Jr. and Donald Hicks. (2004) “The Thermodynamics of DNA Structural Motifs”, Annu. Rev. Biophys. Biomol. Struct. 33, 415-40.

The sequence-specific primers described above and throughout this specification may be prepared using any suitable method, such as, for example, the known, phosphotriester and phosphite triester methods, or automated embodiments thereof. In one such automated embodiment, dialkyl phosphoramidites are used as starting materials and may be synthesized as described by Beaucage et al. Tetrahedron Letters (1981) 22:1859. Methods for synthesizing oligonucleotides on a modified solid support are described, e.g., in U.S. Pat. No. 4,458,066 and published U.S. Application Nos. 20070037175 and 20070059692. It is also possible to use a primer that has been isolated from a biological source (such as the cleaved products of a restriction endonuclease digest).

As indicated above, in generating labeled target nucleic acids according to some embodiments of the subject methods, the above-described genomic template and sequence-specific primers are employed together in a primer extension reaction that produces the desired labeled target nucleic acids. Primer extension reactions for generating labeled nucleic acids are well known to those of skill in the art, and any convenient protocol may be employed, so long as the above described genomic template and sequence-specific primers are employed. In this step of the subject methods, the primer is contacted with the template under conditions sufficient to extend the primer and produce a primer extension product. As such, the above primers are contacted with the genomic template in the presence of a sufficient DNA polymerase under primer extension conditions sufficient to produce the desired primer extension molecules. Any suitable polymerase may be used in the primer extension reaction. DNA polymerases of interest include, but are not limited to, polymerases derived from E. coli, thermophilic bacteria, archaebacteria, phage, yeasts, Neurosporas, Drosophilas, primates and rodents, likewise they include polymerases such as Reverse Transcriptases and the like. In some embodiments, Klenow polymerase is used. In some embodiments, a mid-thermophilic DNA polymerase can be used, a non-limiting example of which is Bst (at a temperature of approximately 50° C.). Non-limiting examples of suitable polymerase include: DNA Polymerase I (E. coli), DNA Polymerase I Large (Klenow) Fragment; Klenow Fragment (3′→5′ exo⁻), Exo-Klenow (3′→5′ exo⁻, 5′→3′ exo⁻); phi29 DNA Polymerase; SP6 RNA Polymerase; T4 DNA Polymerase; T7 DNA Polymerase (unmodified). These and other suitable polymerases are available commercially (e.g., from New England Biolabs; neb.com/nebecomm/products/category6.asp?#9). The DNA polymerase extends the primer according to the genomic template to which it is hybridized in the presence of additional reagents which include, but are not limited to: dNTPs; monovalent and divalent cations, e.g. KCl, MgCl₂; sulfhydryl reagents, e.g. dithiothreitol; and buffering agents; e.g. Tris-Cl.

The length of target nucleic acid, or range of lengths, can be selected by use of suitable reaction stopping conditions. Examples of such conditions include the addition of a chelator (such as EDTA), the addition of di-deoxy nucleotides, or the use or heat (for non-thermophilic polymerases). Suitable conditions for terminating the reaction can be determined empirically. In some embodiments, the range of lengths of the primer extension product is in the range of about 10 to 50, 20 to 200, 30 to 150, 50 to 100, or 500 to 1000 nucleotides.

In some embodiments, the reagents employed in the subject primer extension reactions include a labeling reagent, where the labeling reagent is often a labeled oligonucleotide, which may be labeled with a directly or indirectly detectable label. A directly detectable label is one that can be directly detected without the use of additional reagents, while an indirectly detectable label is one that is detectable by employing one or more additional reagent, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g. dCTP. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels may also be employed as are known in the art.

In some embodiments of the primer extension reactions employed in the subject methods, the genomic template can be first subjected to strand disassociation condition, e.g., subjected to a temperature ranging, e.g., from about 80° C. to about 100° C., usually from about 90° C. to about 95° C. for a period of time, and the resultant disassociated template molecules are then contacted with the primer molecules under annealing conditions, where the temperature of the template and primer composition is reduced to an annealing temperature in the range of from about 20° C. to about 80° C., usually from about 37° C. to about 65° C. In some embodiments, a “snap-cooling” protocol is employed, where the temperature is reduced to the annealing temperature, or to about 4° C. or below in a period of from about 1 second to about 30 seconds, usually from about 5 seconds to about 10 seconds. The sequence-specific primers can be designed as described above for optimal binding under the conditions of the primer extension reaction.

The resultant annealed primer/template hybrids are then maintained in a reaction mixture that includes the above-discussed reagents at a sufficient temperature and for a sufficient period of time to produce the desired labeled target nucleic acids. In some embodiments, this incubation temperature can range from about 20° C. to about 75° C., usually from about 37° C. to about 65° C. In some embodiments, the incubation time can range from about 5 min to about 18 hr, usually from about 1 hr to about 12 hr. for example.

The above protocol results in the production of labeled template nucleic acids. Where desired, the resultant produced labeled template nucleic acids may be separated from the remainder of the reaction mixture, where any convenient separation protocol may be employed.

In some embodiments, the methods result in the production of a select population of labeled template nucleic acids corresponding to genes and more specifically coding regions within genes from an initial genomic template.

In some embodiments, there are provided methods for comparing populations of nucleic acids and compositions for use therein, where a characteristic of the methods is the use of a population of distinct substrate immobilized oligonucleotide features, e.g., an array of substrate immobilized oligonucleotide features. In some embodiments of such methods, the first step is to provide at least two different populations or collections of nucleic acids that are to be compared. The two or more populations of nucleic acids may or may not be labeled, depending on the particular detection protocol employed in a given assay. For example, in some embodiments, binding events on the surface of a substrate may be detected by means other than by detection of a labeled nucleic acid, such as by change in conformation of a conformationally labeled immobilized oligonucleotide, detection of electrical signals caused by binding events on the substrate surface, etc. In many embodiments, however, the populations of nucleic acids are labeled, where the populations may be labeled with the same label or different labels, depending on the actual assay protocol employed. For example, where each population is to be contacted with different but identical arrays, each nucleic acid population or collection may be labeled with the same label. Alternatively, where both populations are to be simultaneously contacted with a single array of immobilized oligonucleotide features, i.e., co-hybridized to the same array of immobilized nucleic acid feature, solution-phase collections or populations of nucleic acids that are to be compared are generally distinguishably or differentially labeled with respect to each other.

The two or more (i.e., at least first and second, where the number of different collections may, in some embodiments, be three, four or more) populations of nucleic acids are prepared from different genomic sources. As such, the first step in some embodiments of the subject methods is to prepare a collection of nucleic acids, e.g., labeled nucleic acids, from an initial genomic source for each genome that is to be compared.

The term “genome” refers to all nucleic acid sequences (coding and non-coding) and elements present in or originating from any virus, single cell (prokaryote and eukaryote) or each cell type and their organelles (e.g. mitochondria) in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

By “genomic source” is meant the initial nucleic acids that are used as the original nucleic acid source from which the solution phase nucleic acids are produced, e.g., as a template in the labeled solution phase nucleic acid generation protocols described in greater detail below.

The genomic source may be prepared using any convenient protocol as described above. In many embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in many embodiments of interest, genomic DNA representing the entire genome from a particular organism, tissue or cell type. However, in some embodiments the genomic source may comprise a portion of the genome, e.g., one or more specific chromosomes or regions thereof, such as PCR amplified regions produced with a pairs of specific primers.

A given initial genomic source may be prepared from a subject, for example a plant or an animal, which subject is suspected of being homozygous or heterozygous for a deletion or amplification of a genomic region. In some embodiments, the average size of the constituent molecules that make up the initial genomic source typically have an average size of at least about 1 Mb, where a representative range of sizes is from about 50 to about 250 Mb or more, while in some embodiments, the sizes may not exceed about 1 MB, such that they may be about 1 Mb or smaller, e.g., less than about 500 Kb, etc.

In some embodiments, the genomic source is “mammalian”, where this term is used broadly to describe organisms which are within the class mammalia, including the orders carnivore (e.g., dogs and cats), rodentia (e.g., mice, guinea pigs, and rats), and primates (e.g., humans, chimpanzees, and monkeys), where of particular interest in some embodiments are human or mouse genomic sources. In some embodiments, a set of nucleic acid sequences within the genomic source is complex, as the genome containsat least about 1×10⁸ base pairs, including at least about 1×10⁹ base pairs, e.g., about 3×10⁹ base pairs.

Where desired, the initial genomic source may be fragmented in the generation protocol, as desired, to produce a fragmented genomic source, where the molecules have a desired average size range, e.g., up to about 10 Kb, such as up to about 1 Kb, where fragmentation may be achieved using any convenient protocol, including but not limited to: mechanical protocols, e.g., sonication, shearing, etc., chemical protocols, e.g., enzyme digestion, etc.

Where desired, the initial genomic source may be amplified as part of the solution phase nucleic acid generation protocol, where the amplification may or may not occur prior to any fragmentation step.

Following provision of the initial genomic source, and any initial processing steps (e.g., fragmentation, amplification, etc.) as described above, the collection of solution phase nucleic acids is prepared for use in the subject methods.

As described above, the collection or population of nucleic acids that is prepared in this step of the subject methods is one that is labeled with a detectable label.

In some embodiments, the initial genomic source, which most often is fragmented (as described above), is employed in the preparation of labeled nucleic acids as a genomic template from which the labeled nucleic acids are enzymatically produced. Different types of template dependent labeled nucleic acid generation protocols are known in the art. In some embodiments, the template is employed in a non-amplifying primer extension nucleic acid generation protocol. In some embodiments, the template is employed in an amplifying primer extension protocol.

In some embodiments, using the above protocols, at least a first collection of nucleic acids and a second collection of nucleic acids are produced from two different genomic sources, e.g., a reference and test genomic template. As indicated above, depending on the particular assay protocol (e.g., whether both populations are to be hybridized simultaneously to a single array or whether each population is to be hybridized to two different but substantially identical, if not identical, arrays) the populations may be labeled with the same or different labels. As such, a characteristic of some embodiments is that the different collections or populations of produced labeled nucleic acids can all be labeled with the same label, such that they are not distinguishably labeled. In some embodiments, a characteristic of the different collections or populations of produced labeled nucleic acids is that the first and second labels can be distinguishable from each other. The constituent members of the above produced collections typically range in length from about 100 to about 10,000 nt, such as from about 200 to about 10,000 nt, including from about 100 to 1,000 nt, from about 100 to about 500 nt, from 100 to 50,000 nt, from 100 to 1,000,000 nt, etc.

In the next step of the subject methods, the collections or populations of labeled nucleic acids produced by the subject methods are contacted to a plurality of different surface immobilized elements (i.e., features) under conditions such that nucleic acid hybridization to the surface immobilized elements can occur. The collections can be contacted to the surface immobilized elements either simultaneously or serially. In many embodiments the compositions are contacted with the plurality of surface immobilized elements, e.g., the array of distinct oligonucleotides of different sequence, simultaneously. Depending on how the collections or populations are labeled, the collections or populations may be contacted with the same array or different arrays, where when the collections or populations are contacted with different arrays, the different arrays are substantially, if not completely, identical to each other in terms of feature content and organization.

The choice of surface immobilized nucleic acids to use may be influenced by prior knowledge of the association of a particular chromosome or chromosomal region with certain disease conditions. International Application WO 93/18186 provides a list of chromosomal abnormalities and associated diseases, which are described in the scientific literature. Alternatively, whole genome screening to identify new regions subject to frequent changes in copy number can be performed using the methods of the present invention. In some embodiments, surface immobilized elements or features can contain nucleic acids representative of locations distributed over the entire genome. The spacing between different locations of the genome that are represented in the features of the collection of features may also vary, and may be uniform, such that the spacing is substantially the same, if not the same, between sampled regions, or non-uniform, as desired.

Of interest are both coding and non-coding genomic regions, (as well as regions that are transcribed but not translated), whereby coding region is meant a region of one or more exons that is transcribed into an mRNA product and from there translated into a protein product, while by non-coding region is meant any sequences outside of the exon regions, where such regions may include regulatory sequences, e.g., promoters, enhancers, introns, inter-genic regions, etc. In some embodiments, one can have at least some of the features directed to non-coding regions and others directed to coding regions. In some embodiments, one can have all of the features directed to non-coding sequences. In some embodiments, one can have all of the features directed to, i.e., corresponding to, coding sequences.

In some embodiments of the subject methods, the copy number of particular nucleic acid sequences in two solution phase collections are compared by hybridizing the collections to one or more nucleic acid, specifically oligonucleotide, arrays, as described above. The hybridization signal intensity, and the ratio of intensities, read from any resultant surface immobilized nucleic acid duplexes (made up of hybridized feature oligonucleotides and solution phase nucleic acids) produced is determined. Since signal intensities on a feature can be influenced by factors other than the copy number of a solution phase nucleic acid population, for some embodiments an analysis is conducted where two labeled populations are present with distinct labels. Thus comparison of the signal intensities for a specific surface immobilized element permits a direct comparison of copy number for a given sequence. Different surface immobilized elements will reflect the copy numbers for different sequences in the solution phase populations. The comparison can reveal situations where each sample includes a certain number of copies of a sequence of interest, but the numbers of copies in each sample are different. The comparison can also reveal situations where one sample is devoid of any copies of the sequence of interest, and the other sample includes one or more copies of the sequence of interest.

As indicated above, hybridization is carried out under suitable hybridization conditions, which may vary in stringency as desired. In some embodiments, highly stringent hybridization conditions may be employed. Representative high stringency assay conditions that may be employed in these embodiments are provided above.

The above hybridization step may include agitation of the immobilized features and the sample of solution phase nucleic acids, where the agitation may be accomplished using any convenient protocol, e.g., shaking, rotating, spinning, and the like.

The hybridization of the labeled target nucleic acids to the probes are then detected using standard techniques. Such applications can compare the copy numbers of sequences capable of binding to the probes. Variations in copy number detectable by the disclosed methods may arise in different ways. For example, copy number may be altered as a result of amplification or deletion of a chromosomal region. Alternatively, copy number may be reduced by genetic rearrangements that alter the sequences in the template nucleic acids sufficiently to reduce binding of the resulting labeled target nucleic acids.

As such, the method can be used for mutation detection, such as for the analysis of multiple gene loci, for example in molecular breeding programs, or in the mapping or identification of genes responsible for polygenic traits.

In some embodiments, previously mapped clones from a particular chromosomal region of interest are used as template. Such clones are becoming available as a result of rapid progress of the worldwide initiative in genomics. Mapped clones can be prepared from libraries constructed from single chromosomes, multiple chromosomes, or from a segment of a chromosome. Standard techniques are used to clone suitably sized fragments in vectors such as cosmids, yeast artificial chromosomes (YACs), bacterial artificial chromosomes (BACs) and P1 phage. While it is possible to generate clone libraries, as described above, libraries spanning entire chromosomes are also available commercially. For instance, chromosome-specific libraries from the human and other genomes are available for Clontech (South San Francisco, Calif.) or from The American Type Culture Collection (see, ATCC/NIH Repository of Catalogue of Human and Mouse DNA Probes and Libraries, 7th ed. 1993). If necessary, clones described above may be genetically or physically mapped. For instance, FISH and digital image analysis can be used to localize cosmids along the desired chromosome. This method is described, for instance, in Lichter et al., Science (1990) 247:64-69. The physically mapped clones can then be used to more finally map a region of interest identified using CGH or other methods.

The probes employed in the subject methods are immobilized on a solid support. Many methods for immobilizing nucleic acids on a variety of solid surfaces are known in the art. For instance, the solid surface may be a membrane, glass, plastic, or a bead. The desired component may be covalently bound or noncovalently attached through nonspecific binding. The immobilization of nucleic acids on solid surfaces is discussed more fully below.

A wide variety of organic and inorganic polymers, as well as other materials, both natural and synthetic, may be employed as the material for the solid surface. Illustrative solid surfaces include nitrocellulose, nylon, glass, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive, materials, cermets or the like. In addition substances that form gels can be used. Such materials include proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, enhance signal detection or the like.

If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. For example, methods for immobilizing nucleic acids by introduction of various functional groups to the molecules is known (see, e.g., Bischoff et al., Anal. Biochem. 164:336 344 (1987); Kremsky et al., Nuc. Acids Res. 15:2891 2910 (1987)). Modified nucleotides can be placed on the target using PCR primers containing the modified nucleotide, or by enzymatic end labeling with modified nucleotides.

Use of membrane supports (e.g., nitrocellulose, nylon, polypropylene) for the nucleic acid arrays of the invention is advantageous in some embodiments because of well developed technology employing manual and robotic methods of arraying targets at relatively high element densities (e.g., up to 30 40/cm²). In addition, such membranes are generally available and protocols and equipment for hybridization to membranes is well known. Many membrane materials, however, have considerable fluorescence emission, where fluorescent labels are used to detect hybridization.

To optimize a given assay format one of skill can determine sensitivity of fluorescence detection for different combinations of membrane type, fluorochrome, excitation and emission bands, spot size and the like. In addition, low fluorescence background membranes have been described (see, e.g., Chu et al., Electrophoresis (1992) 13:105-114).

The sensitivity for detection of spots of various diameters on the candidate membranes can be readily determined by, for example, spotting a dilution series of fluorescently end labeled DNA fragments. These spots are then imaged using conventional fluorescence microscopy. The sensitivity, linearity, and dynamic range achievable from the various combinations of fluorochrome and membranes can thus be determined. Serial dilutions of pairs of fluorochrome in known relative proportions can also be analyzed to determine the accuracy with which fluorescence ratio measurements reflect actual fluorochrome ratios over the dynamic range permitted by the detectors and membrane fluorescence.

Arrays on substrates with much lower fluorescence than membranes, such as glass, quartz, or small beads, can achieve much better sensitivity. For example, elements of various sizes, ranging from the about 1 mm diameter down to about 1 μm can be used with these materials. Small array members containing small amounts of concentrated probe DNA are conveniently used for high complexity comparative hybridizations since the total amount of target available for binding to each element will be limited. Thus, in some embodiments, it is advantageous to have small array members that contain a small amount of concentrated probe DNA so that the signal that is obtained is highly localized and bright. Such small array members are typically used in arrays with densities greater than 10⁴/cm². Relatively simple approaches capable of quantitative fluorescent imaging of 1 cm² areas have been described that permit acquisition of data from a large number of members in a single image (see, e.g., Wittrup et. al. Cytometry 16:206-213 (1994)).

Covalent attachment of the probe nucleic acids to glass or synthetic fused silica can be accomplished according to a number of known techniques. Such substrates provide a very low fluorescence substrate, and a highly efficient hybridization environment.

There are many possible approaches to coupling nucleic acids to glass that employ commercially available reagents. For instance, materials for preparation of silanized glass with a number of functional groups are commercially available or can be prepared using standard techniques. Alternatively, quartz cover slips, which have at least 10-fold lower auto fluorescence than glass, can be silanized.

The probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith et al. Science, 258:1122 1126 (1992)). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

The copy number of particular nucleic acid sequences in two target collections prepared according to the subject methods are compared by hybridizing the targets to one or more probe nucleic acid arrays, as described above. The hybridization signal intensity, and the ratio of intensities, produced by the targets on each of the probe elements (i.e., features) is determined. Since signal intensities on a probe element can be influenced by factors other than the copy number of a target in solution, in some embodiments, an analysis can be conducted where two labeled populations are present with distinct labels. Thus comparison of the signal intensity ratios among probe elements permits comparison of copy number ratios of different sequences in the target populations.

Standard hybridization techniques are used in nucleic acid array analysis. Suitable methods are described in references describing CGH techniques (Kallioniemi et al., Science (1992) 258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. (1981) Meth. Enzymol., 21:470-480 and Angerer et al. in Genetic Engineering: Principles and Methods Setlow and Hollaender, Eds. Vol 7, pgs 43 65 (plenum Press, New York 1985). See also U.S. Pat. Nos. 6,335,167; 6,197,501; 5,830,645; and 5,665,549; the disclosures of which are herein incorporate by reference.

Generally, nucleic acid hybridizations comprise the following major steps: (1) immobilization of probe nucleic acids; (2) prehybridization treatment to increase accessibility of target DNA, and to reduce nonspecific binding; (3) hybridization of the mixture of nucleic acids to the nucleic acid on the solid surface; (4) posthybridization washes to remove nucleic acid fragments not bound in the hybridization and (5) detection of the hybridized nucleic acid fragments. The reagent used in each of these steps and their conditions for use vary depending on the particular application.

Reading of the resultant hybridized array may be accomplished by illuminating the array and reading the location and intensity of resulting fluorescence at each feature of the array to detect any binding complexes on the surface of the array as described above.

Results from the reading or evaluating may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample, or whether or not a pattern indicates a particular condition of an organism from which the sample came).

In some embodiments, the subject methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Analysis of processed results of the described hybridization experiments provides information about the relative copy number of nucleic acid domains, e.g. genes, in genomes.

### Kits

Also provided are kits for use in the subject methods, where such kits may comprise containers, each with one or more of the various reagents (typically in concentrated form) utilized in the methods, where such reagents include, but are not limited, the subject sequence-specific primers, buffers, the appropriate nucleotide triphosphates (e.g. dATP, dCTP, dGTP, dTTP), polymerase, labeling reagents, e.g., labeled nucleotides, and the like. Where the kits are specifically designed for use in applications such as CGH, the kits may further include labeling reagents for making two or more collections of distinguishably labeled nucleic acids according to the subject methods. Kit can comprise an array of probe nucleic acids as described herein, hybridization solution, etc.

The kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

The above disclosure demonstrates that novel methods of producing labeled target nucleic acids from genomic template is provided, where advantages of the subject methods include the feature that the produced populations are less complex than target populations produced by other methods, such as nick translation or random primer extension, and are therefore more suitable for use with immobilized probe array based CGH applications. As such, the subject methods represent a significant contribution to the art.

Although the foregoing has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

What is claimed is:

1. A method for evaluating a region of interest in a genomic template, the method comprising:
a) contacting a genomic template with a sequence-specific primer under conditions to perform a template-dependent primer-extension reaction,
b) extending said primer into said region of interest to form a target nucleic acid comprising a probe-binding sequence,
c) contacting said target with a nucleotide array, said array comprising a feature comprising a probe complementary to said probe-binding sequence, and
d) determining binding between said feature and said target.

2. The method of claim 1 wherein said region of interest comprises a coding region.

3. The method of claim 1 wherein said target is labeled with a detectable label.

4. The method of claim 1 wherein the sequence of said primer is selected based on the known sequence of said template.

5. The method of claim 1 wherein the sequence of said probe-binding region is selected based on the known sequence of said template.

6. The method of claim 1 wherein the sequence of said primer and the sequence of said probe-binding region are selected based on the known sequence of said template.

7. The method of claim 1 wherein said primer binds downstream of said region of interest.

8. The method of claim 1 wherein said evaluating comprising determining the copy number of said region of interest.

9. The method of claim 1 wherein said primer binds at a location about 10 to 100 nucleotides downstream of said region of interest.

10. The method of claim 1 wherein said target comprises an intervening sequence between said primer and said probe-binding sequence, wherein said intervening sequence is in the range of 10 to 1000 nucleotides.

11. The method of claim 1 wherein,
step (a) comprises contacting the genomic template with a plurality of sequence-specific primers under conditions to perform template-dependent primer-extension,
step (b) comprises extending said primers into said region of interest to form a plurality of target nucleic acids, each comprising a unique probe-binding sequence,
step (c) comprises contacting said targets with a nucleotide array, said array comprising a plurality of features, wherein each said unique probe-binding sequence is complementary to a unique feature of said array, and
step (d) comprises determining binding between said unique features and said target nucleic acids.

12. A method for evaluating a plurality of regions of interest in a genomic template, the method comprising:
a) contacting a genomic template with a plurality of sequence-specific primers under conditions to perform template-dependent primer-extension,
b) extending said plurality of primers into said regions of interest to form target nucleic acids, each comprising a unique probe-binding sequence, wherein at least one target nucleic acid is formed corresponding to each region of interest,
c) contacting said target nucleic acids with a nucleotide array, said array comprising a plurality of unique features, wherein each unique probe-binding sequence is complementary to a unique feature of said array, and
d) determining binding between said unique features and said target nucleic acids.

13. The method of claim 12 wherein said regions of interest comprise a plurality of coding regions.

14. The method of claim 12 wherein the number of primers for each of said regions of interest is in the range of between 10 and 100.

15. The method of claim 12 wherein the number of regions of interest is at least 1,000.

16. The method of claim 12 wherein the number of regions of interest is at least 100,000.

17. The method of claim 12 wherein said probe-binding sequences have melting temperatures within a range of 6° C.,

18. A method for comparing the relative copy number of nucleic acid sequences in two or more collections of nucleic acid molecules, the method comprising: (a) preparing at least a first collection of labeled nucleic acid target molecules labeled with a first label and a second collection of labeled nucleic acid target molecules labeled with a second label distinguishable from said first label, wherein each constituent member of said first and second collections of labeled nucleic acid target molecules is prepared from a genomic nucleic acid template using a set of sequence-specific primers; (b) contacting said first and second collections of labeled nucleic acid target molecules with a plurality of features bound to a solid surface; (c) evaluating the relative binding of the first and second collections of labeled nucleic acid target molecules to the solid surface to compare the relative copy number of nucleic acid sequences in said first and second collections of labeled nucleic acid target molecules.

19. The method of claim 18, wherein said plurality of features bound to said solid surface comprise an array.

20. The method of claim 18, wherein the first collection of labeled nucleic acids is from a test genome and the second collection of labeled nucleic acids is from a normal reference genome.

21. The method according to claim 18, wherein said set of primers is not a random set of primers.

22. The method according to claim 18, wherein said method further comprises a data transmission step in which a result from said evaluating is transmitted from a first location to a second location.

23. A method comprising receiving data representing a result of said evaluation obtained by the method of claim 18.

24. A kit for use in evaluating a region of interest in a genomic template, said kit comprising:
(a) a plurality of distinct features bound to a surface of a solid support; and
(b) instructions for practicing the method according to claim 12.

25. The kit according to claim 24, wherein said kit further comprises a set of sequence-specific primers.

26. The kit according to claim 24, wherein said kit further comprises a nucleic acid labeling reagent.

27. The kit according to claim 24, wherein said kit further comprises a polymerase.

28. A method for use in evaluating a region of interest in a genomic template, the method comprising:
a) contacting a genomic template with a sequence-specific primer under conditions to perform a template-dependent primer-extension reaction, and
b) extending said primer into said region of interest to form a target nucleic acid comprising a probe-binding sequence.
